(19) United States
(12) Patent Application Publication
(10) Pub. No.: US 2019/0212332 A1
(43) Pub. Date: Jul. 11, 2019

(54) MlCRO-SCREENING AND SORTING APPARATUS, PROCESS, AND PRODUCTS

(71) Applicants: The Board of Trustees of the Leland Stanford Junior University, Palo Alto, CA (US); Orca Biosystems, Inc., Mountainview, CA (US)

(72) Inventors: Ivan DIMOV, Union City, CA (US); Nathaniel FERNHOFF, Menlo Park, CA (US); Irving WEISSMAN, Stanford, CA (US); Charles Kwok Fai CHAN, Redwood City, CA (US); Everett MEYER, Belmont, CA (US); Peter Lincoln WANG, Menlo Park, CA (US); Bruce RICHARDSON, Los Gatos, CA (US)

(21) Appl. No.: 16/334,353

(22) PCT Filed: Sep. 19, 2017
(86) PCT No.: PCT/US2017/052218

§ 371 Date: Mar. 18, 2019

Related U.S. Application Data
(60) Provisional application No. 62/396,541, filed on Sep. 19, 2016.

(51) Int. Cl. G01N 33/543; C12N 5/074; C12N 5/0775; C12N 5/077; C12N 5/0735; A61P 37/06; A61K 35/12; C12N 5/09; C40B 30/04

(52) U.S. Cl. CPC G01N 33/543; C12N 5/0696; C12N 5/0663; C12N 5/0669; A61K 2035/124; A61P 37/06; A61K 35/12; C12N 5/0693; C40B 30/04; C12N 5/0606

(57) ABSTRACT

The disclosure provides methods and apparatus for high speed and sterile sorting of heterogeneous populations of cells to isolate homogenous populations. In embodiments, the disclosure provides a micropore array and collection tray situated within a sterile, closed cartridge.

### BACKGROUND

### Field

The disclosure is directed to apparatus and methods useful for the identification, selection, and sorting of cells having therapeutic applications.

### Background

High-throughput measurements have begun to provide insight into the intrinsic complexities and dense interconnectivities of biological systems. As examples, whole-genome sequencing has yielded a wealth of information on crucial genes and mutations underlying disease pathophysiology, DNA microarrays have allowed transcription patterns of various cancers to be dissected, and large-scale proteomics methods have facilitated the study of signaling networks in cells responding to various growth factors. In addition, high throughput micropore arrays have been described and are useful for cell-based applications, including protein engineering or production of other products within cells.

Related to these methods and products are high throughput cell sorting technologies that facilitate isolating populations of cells for various downstream applications. For example, cell sorting is useful for characterizing the expression pattern of various endogenous or exogenous products, such as proteins, within cells. In some applications, the expression of products within cells provide one or more markers that are characteristic of populations of cells with desirable physiologic properties. Other applications of high throughput cell sorting technologies comprise isolating populations of cells with useful therapeutic properties.

Cell-based therapies represent a cornerstone of regenerative medicine and immunotherapies. Healthy stem cells can replace the damaged tissue of a patient, and specific immune cells can cure cancer and autoimmune diseases. In both of these instances, the therapeutic effect is derived from a defined population of cells, but the biological source material for these cells is heterogeneous because it is comprised of multiple different cell types. While many of the non-therapeutic cells contaminating the therapeutically relevant cells are harmless, even a small population of a specific errant cell type can cause severely adverse consequences in the recipient. For example, residual tumor cells, or teratoma initiating cells, that contaminate a population of transplanted cells can seed a tumor in a patient. In another example, subsets of circulating T cells can initiate graft-versus-host-disease (GVHD), thereby counteracting the therapeutic benefits of other T-cells introduced during transplantation. Therefore, it is necessary to purify the therapeutic cells away from the deleterious cells before transplanting the cells into a patient. To that end, there is a need for a high-throughput, high-purity method to isolate rare stem cells and other immune cell types based on differential surface marker expression in a sterile and clinically applicable format.

Fluorescently activated cell sorting (FACS) is one high-throughput method for purifying or sorting cell populations. (Herzenberg, L. A. et al. Clin. Chem. 48, 1819-1827 (2002).) FACS has applications in immunology, cancer, stem cell biology, and protein engineering, making it possible to purify rare cells with good viability from large heterogeneous cellular mixtures. Widespread use of FACS has pushed the technique to its performance limits, and there is a need in the art for improved cell purification and sorting methods. Furthermore, the clinical application of FACS is limited by two primary obstacles: insufficient speed of cell sorting, and lack of sterility during the sorting process.

Certain limitations of the FACS method are illustrated by bone marrow or peripheral blood transplantation. In allogeneic transplantation (i.e. wherein the cells or tissues are from a genetically similar, but not identical, donor), T-cells in the transplant cause graft-versus-host disease (GVHD). (Blazar, B. R., Murphy, W. J. & Abedi, M., Nat. Rev. Immunol. 12, 443-458 (2012).) A T-cell frequency as low as 0.0014% (i.e., 50,000 T-cells/kg) in the transplant poses considerable risk that acute or chronic GVHD can develop. (Alters, S. E. et al., J. Exp. Med. 173, 491-494 (1991); Slaper-Cortenbach, I. C. M. et al., Rheumatology 38, 751-754 (1999).) Thus, there is a need in the art for methods that effectively isolate a population of cells enriched for only those cell-types with desirable characteristics.

In autologous transplantation (i. e. wherein the cells or tissues are taken from the recipient), bone marrow and peripheral blood samples can be contaminated with tumor forming cells. (Dick, F., Bloomfield, C. D. & Brunning, R. D., Cancer 33, 1382-1398 (1974); Stein, R. S. et al., Cancer 37, 629-636 (1976); Uckun, F. M. et al., Blood 69, 361-366 (1987); Takvorian, T. et al., N. Engl. J. Med. 316, 1499-1505 (1987).) Thus, therapeutic administration of isolated, purified hematopoietic stem cells (HSCs) that bare orders of magnitude fewer contaminating tumor initiating cells extends progression-free patient survival by 12-14 years. (Muller, A. M. S. et al., Biol. Blood Marrow Transplant. J. Am. Soc. Blood Marrow Transplant. 18, 125-133 (2012).) As this example also illustrates, there is a need in the art for methods and apparatus capable of isolating therapeutically relevant cell populations away from contaminating cells detrimental to patient outcomes, or contaminating cells that interfere with other downstream applications.

To eliminate detrimental and unwanted outcomes, such as GVHD and neoplasm recurrence, harmful cells should be depleted below the effector threshold from the transplant. However, with 2.8 billion cells in an average transplant, FACS technology would require more than 24 hours to sort the cells. Such long sorting and supervision times negatively affect the primary cell functionality and viability. In addition, the long operator sorting times make scaling the therapies to thousands of patients practically unfeasible.

Additionally, FACS technology was not designed to meet clinical criteria for sterility. Samples from separate patients interact with the same fluidic surfaces. Thus, extensive time and labor is required for sterilization. Furthermore, all FACS samples are aerosolized into micro-droplets for sorting the cells, creating additional biohazard risks for the operator. Thus FACS is not readily amenable to clinical applications where sterility is of paramount importance.

Outside of the realm of hematopoietic cell transplantation, other fields of regenerative medicine require high-throughput, sterile sorting of tissue-specific stem cells. For example, a commonly proposed paradigm of regenerative medicine requires differentiation of embryonic stem cells, or induced pluripotent stem cells (iPSCs), to tissue-specific cells: a process that inefficiently produces large heterogeneous populations with undesirable (e.g., teratoma-forming) cells from which large numbers (10⁴-10⁶ cells/kg) of safely transplantable cells must be purified. Again, therapeutic use requires contaminating impurities be removed below the threshold dose that causes an adverse event.

Alternative techniques for purifying and sorting cells also present disadvantages for which novel solutions are necessary. For example, magnetic activated cell sorting (MACS) may address the speed and sterility issues associated with FACS methods. However, in each case non-specific interactions prevent these techniques from reliably depleting contaminating and otherwise undesirable cell populations below the desired or required effector threshold.

In the case of MACS, the method is capable of enriching clinically relevant numbers of cells in a timely fashion, and the fluidics surfaces that contact the biological sample are exchangeable and disposable in MACS. These features address the speed and sterility issues of FACS. However, what is gained in speed and sterility is lost in purity and functionality. MACS enrichments generally yield 70%-95% purity compared to >99% by FACS. Thus, there is a need in the art for methods and apparatus that achieve the level of purity achieved using FACS, but with the speed and sterility achievable by MACS methods.

Of equal importance, MACS is only capable to sort for a single marker: cells are either bound to a magnetic particle or not. This approach precludes multi-parameter interrogation. Therefore, the purity of cells after MACS separation is generally assessed only in relation to that single marker rather by the purity of a functional class of cells. For instance, all T cells may be depleted using the CD3 marker, however the separation of various T cells subsets responsible for adverse and therapeutic effect require use of at least 4 markers. Thus, there is a need in the art for methods and apparatus that provide multi-parameter cell purification and sorting that is limited by, or entirely absent from, existing apparatus or method. Furthermore, no existing apparatus or method is capable of achieving multi-parameter cell purification and sorting with the speed and sterility required for clinical applications.

The problem of GVHD during cell transplantation illustrates the significance of, and need for, multi-parameter cell purification and sorting methods. Donor T cells are both required for, and detrimental to, engraftment. This is because GVHD, a detriment to engraftment, results from the activation of a subset of donor T cells against the normal, healthy tissue of the recipient. At the same time, however, donor T cells also help the transplanted cells to engraft, exert an anti-tumor effect, and provide protection from infection. A solution to this conundrum is afforded by the discovery that two different sub-populations of T cells are responsible for the detrimental and beneficial effects. Specifically, naïve T cells mediate the detrimental effects, whereas memory T cells promote engraftment. However, the art lacks a clinically feasible method to purify the desirable sub-population of T cells from one another, while simultaneously purifying the T cell population from the remained of heterogeneous donor cell-types.

Thus, novel and scalable technologies for cell sorting and purification are necessary: (i) to improve therapeutic cell transplantation, which is currently used for the treatment of over 100 diseases (Atkinson, K. Clinical Bone Marrow and Blood Stem Cell Transplantation. (Cambridge University Press, 2004)); and (ii) to enable stem or progenitor cell transplantation, which is the basis of regenerative medicine. (Passier, R., van Laake, L. W. & Mummery, C. L., Nature 453, 322-329 (2008)).

### SUMMARY

In one aspect, the disclosure is directed to a method for sorting a heterogeneous starting population of cells having a plurality of phenotypes. The method includes the following steps: loading a micropore array with a starting population of cells; imaging the micropore array to identify individual pores comprising cells having a phenotype of interest; extracting cells having a phenotype of interest from the individual pores by directing electromagnetic radiation at a radiation absorbing material associated with the pores, and; collecting extracted cells having a phenotype of interest, wherein the scanning, imaging, and extracting steps sort between 2×109 and 4×109 micropores within 120 minutes.

In another aspect, the rapid extraction of the method of the disclosure is achieved by using a polygon scanning system. For instance, in an embodiment of the disclosure the system includes micropore array; an electromagnetic radiation source, a rotating polygon mirror, an F-theta lens, wherein the mirror and the lens scan the focus of the electromagnetic radiation source at micropores of the array at a rate of greater than 150,000 micropores per second, and a detector for detecting electromagnetic radiation from the pores of the array.

In the various aspects of the disclosure, one or more phenotypic markers in cells of the starting population can be detected with binding agents capable of detecting a phenotype or phenotypes of interest.

In addition, the micropore array is enclosed in a sorting cartridge comprising a sterile housing. The housing may prevent the starting population of cells or the extracted cells from contacting of a sorting instrument during use.

In some aspects, the starting population of cells includes bone marrow cells, peripheral hematopoietic cells, differentiated ES cells, iPSCs, or genetically modified cells. The extracted cells may include a population suitable for allogeneic or autologous transplantation into a subject. For instance, the starting population of cells may include heterogeneous peripheral hematopoietic cells, and the extracted cells comprise a population of purified hematopoietic cells with reduced naïve T-cells. The population of purified hematopoietic cells may result in reduced or undetectable incidence of graft-versus-host-disease when transplanted into a subject. In one embodiment, the starting population of cells includes a heterogeneous population containing tumorigenic or teratogenic cells, and the extracted cells include a population of purified cells free substantially or completely lacking tumorigenic or teratogenic cells. In another embodiment, the starting population of cells includes a heterogeneous population of differentiated Embryonic Stem Cells, or a heterogeneous population of differentiated induced Pluripotent Stem Cells, and the extracted cells include a homogenous population of differentiated cells. Still further, the starting population of cells may include a heterogeneous population of cells subjected to genetic modification, and the extracted cells may include a homogenous population of cells subjected to genetic modification.

In various aspects of the apparatus and method of the disclosure, at least 10⁵ cells are scanned and imaged simultaneously. For instance, the scanning, imaging, and extracting steps can be performed at a rate of at least about 500×105 cells/sec. In some aspects, less than or equal to 1 cell is loaded into to each pore of the micropore array. Furthermore, the micropore array may include particles comprising a radiation absorbing material adhered to the interior walls of the pores and the extracting cells by directing electromagnetic radiation at radiation absorbent material associated with the pores involves a 1 nsec, 90 μJ laser pulse.

### BRIEF DESCRIPTION OF THE FIGURES

FIG. 1 shows a schematic representation of an example method of micropore cell sorting according to the disclosure.

FIG. 2, Panels A-D shows fluorescent microscope images of cells loaded at 0.5(A), 1(B), 5 (C) and 10 (D) cells per pore into a 100 μm diameter micropore array. FIG. 2, Panel E shows a representative raw fluorescent image of a 20 μm diameter pore array loaded with GFP expressing cells (punctate staining pattern). FIG. 2, Panel F shows a scatter plot after automatically scanning and analyzing 0.5 million pores from the array in Panel E.

FIG. 3 shows one embodiment of the closed, micropore array cell sorting cartridge according to the disclosure.

FIG. 4, Panels A-G, shows the experimental design of an in vitro viability assay.

FIG. 5, Panels A and B, shows direct comparison between FACS cytometry of cells and micropore array based cytometry of the cells. Axes display relative fluorescence units from each setup.

FIG. 6, Panel A shows a bright field image of a micropore array filled with 2.8 μm magnetic particles before laser extraction. The arrow shows the focal site of the laser and the pore that will be extracted. FIG. 6, Panel B shows the target pore (indicated by the arrow) after the laser pulse. FIG. 6, Panel C shows a GFP MOLM-13 cell targeted for extraction (indicated by the arrow). FIG. 6, Panel D shows the same pore as in Panel 6C after the laser extraction (indicated by the arrow). FIG. 6, Panels E & F show bright-field and green fluorescent micrographs, respectively, of the collection tray under the extracted cell from Panel 6C. FIG. 6, Panel E shows the extracted magnetic beads in the collection tray after extraction. FIG. 6, Panel F shows a GFP positive cell in the collection tray (indicated by the arrow). All scale bars represent 40 μm.

FIG. 7 shows in vitro cell viability after micropore array sorting of an adherent cell line.

FIG. 8 shows that a “SuperGraft” population of cells capable of being produced according to the disclosure exhibits reduced acute GVHD incidence in an animal engraftment model.

FIG. 9 shows myeloablated BALB/c mice injected with the J774 cell line harboring a GFP-luciferase reporter gene to non-invasively detect tumor burden, and subsequently treated with the indicated hematopoietic cell populations from allogeneic C57Bl/6 mice (bottom).

FIG. 10 shows a schematic representation of an ultra-fast laser scanning system according to the disclosure.

### DESCRIPTION

In various embodiments, the disclosure is directed to the identification and segregation of a subpopulation of homogeneous cells large heterogeneous population of cells. The embodiments of the disclosure can be used to generate populations of cells suitable for transplantation into a subject with clinically suitable processing speeds and sterility.

Aspects of the present disclosure provide methods and apparatus for sterile sorting of heterogeneous populations of cells to isolate homogenous cell populations. In various aspects, the disclosure provides a micropore array for cell sorting and a sterile cell collection apparatus. Accordingly, embodiments of the disclosure significantly reduce the probability of sample-to-sample contamination during use, and maintain sterile conditions during processing and sorting of cell populations. Furthermore, embodiments of the disclosure eliminate the escape of aerosolized particles, thus protecting the operator from potentially harmful bio-hazards generated during cell sorting and isolation.

In one aspect, the disclosure is directed to an adaptation of the microcavity-based platform described in U.S. patent application Ser. No. 15/050,130, which is incorporated herein by reference in its entirety. The platform disclosure herein enables isolation of desirable, pure populations of cells on the order of 2×10⁹ and 4×10⁹ cells within 120 minutes.

Accordingly, the disclosure is directed to a strategy for sorting cells using high-density micropore arrays and ultra-fast laser-extraction. In various embodiments, the method may sort rates of over 1 million cells/sec with high purity and sterility. Because a key factor for clinical cell sorting is the sorting rate, the method and apparatus of the disclosure provide micropore sorting that does not require cells to flow through a single channel (as in FACS). In one embodiment, the apparatus includes a polygon scanner for fast remote processing, allowing a laser used for extracting cells from the array to be switched on and off at rates over a million times per second, have its spot location precisely controlled, and achieve linear scan velocities of 2000 meters/min. For example, if the laser spot or pixels are aimed at the micropores and a 20 μm pore-pitch array is used, the laser raster will be able to sort at speeds of 1 million cells per second or faster. This can represent 100-1000 fold faster sorting speeds over conventional FACS technology. The disclosure can provide therapeutic cell sorting applications that require minimal ex vivo processing time and a maximal number of sorted cells. For instance, a biological sample comprised of 1,000,000,000 cells would require greater than 24 hours to sort by FACS (10,000 cells/second), but would require less than 17 minutes for an equivalent process using the apparatus and method of the disclosure.

The apparatus and method of the disclosure allows for the multi-parameter interrogation and sorting of cells. Optical and fluorescent properties of each cell can be measured and recorded and used as a basis for selection. In one embodiment, a laser scanning optics is used so that multiple fluorescent features are analyzed for each cell and then subsets of cells are selected based on a combination of such features.

The use of a static array of cells can make the identification of cells having phenotypes of interest highly accurate and content-rich since there is no flow-rate time-constraint. Single cell extractions can also be verified by imaging the cells in the collection tray. Furthermore cells do not experience any shear stress, except during the microsecond laser extraction. Before sorting, each cell can be imaged and verified multiple times and at multiple magnifications providing very rich information for sorting. In addition, cells can be cultured and secreted proteins analyzed in each pore.

### Definitions

Unless otherwise defined, the technical and scientific terms used herein have the same meaning as commonly understood by one of ordinary skill in the art. Expansion and clarification of some terms are provided herein. All publications, patent applications, patents and other references mentioned herein, if not otherwise indicated, are explicitly incorporated by reference.

As used herein, the singular forms “a,” “an”, and “the” include plural referents unless the context clearly dictates otherwise.

As used herein, the terms “cavity” and “pore,” and “capillary” refer interchangeably to the internal spaces defined by the arrays of the disclosure. Accordingly, a “microcavity array” is equivalent to a “micropore array,” which is equivalent to a “microcapillary array.” Similarly, the disclosure refers at times to the contents of a cavity or cavities, and at other times to the contents of a pore or pores, and a still other times to the content of a capillary or capillaries, which are in all cases equivalent according to the disclosure.

The terms “binding partner”, “ligand” or “receptor” as used herein, may be any of a large number of different molecules, or aggregates, and the terms are used interchangeably. Proteins, polypeptides, peptides, nucleic acids (nucleotides, oligonucleotides and polynucleotides), antibodies, saccharides, polysaccharides, lipids, receptors, test compounds (particularly those produced by combinatorial chemistry), may each be a binding partner.

The term “biological element” as used herein, refers to any biological cell or bioreactive molecule. Non-limiting examples of the bioreactive molecules include proteins, nucleic acids, peptides, antibodies, antibody fragments, enzymes, hormones, and small molecules.

As used herein, the term “heterogeneous” refers to a mixture of elements, such as cells, with a range of phenotypic or physiologic characteristics. For example, a “heterogeneous population of cells” contains a number of different cell types, such as different subsets of T-cells, hematopoietic cells, cells of varying states of differentiation, cells of varying tumorigenic potential, or other parameters of phenotypic or physiological variation that will be recognized by those of skill in the art. Another non-limiting example of a heterogeneous population of cells is a population wherein some, but not all, cells are genetically modified.

In contrast, as used herein, the term “homogeneous” refers to a collection of elements, such as cells, with the same, or similar, phenotypic or physiologic characteristics. For example, a “homogeneous population of cells” contains cells of the same or similar types, such as subsets of T-cells expressing the same or similar phenotypic markers, or other hematopoietic cells that express a common or similar set of phenotypic markers. Similarly, a homogenous population of cells includes cell populations that exhibit the same differentiation state, or the same tumorigenic potential. Likewise, a homogenous population of cells includes a population wherein all, or substantially all, of the cells comprise the same genetic modification.

Furthermore, those skilled in the art will recognize that the terms “heterogeneous” and “homogenous” are terms of degree. Thus, a population of cells that possesses a greater degree of dissimilarity among its constituents is heterogeneous relative to a reference population with less dissimilarity, but the same population may also be a “homogenous” relative to another reference population with greater dissimilarity.

The term “bind” or “attach” as used herein, includes any physical attachment or close association, which may be permanent or temporary. Non-limiting examples of these associations are hydrogen bonding, hydrophobic forces, van der Waals forces, covalent bonding, and/or ionic bonding. These interactions can facilitate physical attachment between a molecule of interest and the analyte being measured. The “binding” interaction may be brief as in the situation where binding causes a chemical reaction to occur, such as for example when the binding component is an enzyme and the analyte is a substrate for the enzyme.

Specific binding reactions resulting from contact between the binding agent and the analyte are also within this definition. Such reactions are the result of interaction of, for example, an antibody and, for example a protein or peptide, such that the interaction is dependent upon the presence of a particular structure (e.g., an antigenic determinant or epitope) on a protein. Specific binding interactions can occur between other molecules as well, including, for example, protein-protein interactions, protein-small molecule interactions, antibody-small molecule interactions, and protein-carbohydrate interactions. Each of these interactions may occur at the surface of a cell.

Turning now to the various aspects of the disclosure, in one aspect, the disclosure provides methods and apparatus for sorting a heterogeneous starting population of cells having a plurality of phenotypes. In an embodiment, the method comprises loading a micropore array with a starting population of cells; imaging the micropore array to identify individual pores comprising cells having a phenotype of interest; extracting cells having a phenotype of interest from the individual pores by directing electromagnetic radiation at a radiation absorbing material associated with the pores, and; collecting extracted cells having a phenotype of interest.

In another aspect, methods of the disclosure include sorting a heterogeneous starting population of cells having a plurality of phenotypes, wherein one or more phenotypic markers in cells of the starting population is detected with binding agents capable of detecting a phenotype or phenotypes of interest. In some embodiments, a binding agent according to the disclosure is an antibody or antibodies. In another embodiment, the binding agent of the disclosure is an antibody fragment, a ligand, a peptide, a small molecule, or a receptor.

In embodiments, the disclosure provides a method of sorting a heterogeneous starting population of cells having a plurality of phenotypes, wherein the starting population of cells is comprised of bone marrow cells, peripheral hematopoietic cells, differentiated ES cells, induced Pluripotent Stem Cells (iPSCs), or genetically modified cells. In some embodiments, the disclosure provides a method of sorting a heterogeneous starting population of cells, wherein cells extracted according to the method comprise a population suitable for transplantation into a subject. For example, the disclosure provides extracted cells comprising a population suitable for allogeneic or autologous transplantation into a subject.

In certain embodiments, the starting population of cells according to the disclosure comprises heterogeneous peripheral hematopoietic cells, and the extracted cells comprise a population of purified hematopoietic cells with reduced naïve T-cells. In some embodiments, the cells extracted according to the disclosure comprise a population of purified hematopoietic cells consisting of less than 0.0014% naïve T-cells. In still further embodiments, the disclosure provides a population of purified hematopoietic cells that show reduced or undetectable incidence of graft-versus-host-disease (GVHD) when transplanted into a subject.

In another aspect, the disclosure provides methods of sorting cells, wherein the starting population of cells comprises a heterogeneous population containing tumorigenic or teratogenic cells, and the extracted cells comprise a population of purified cells free substantially or completely lacking tumorigenic or teratogenic cells. In an alternative aspect, the disclosure provides methods of sorting cells, wherein the starting population of cells comprises a heterogeneous population of differentiated Embryonic Stem Cells, or a heterogeneous population of differentiated induced Pluripotent Stem Cells, and the extracted cells comprise a homogenous population of differentiated cells. In yet another aspect, the disclosure provides methods of sorting cells, wherein the starting population of cells comprises a heterogeneous population of cells subjected to genetic modification, and the extracted cells comprise a homogenous population of cells subjected to genetic modification.

In some embodiments, the methods and apparatus of the disclosure scan, image, and extract from the array at least 10⁵ cells simultaneously. In some aspects, the scanning, imaging, and extracting steps according to the disclosure are performed at a rate of at least about 100,000 cells per second. In another aspect, the scanning, imaging, and extracting steps according to the disclosure sort between 2×10⁹ and 50×10⁹ cells within 120 minutes. The moving laser will excite each pore for between 50-1000 ns, meaning a focused spot speed of 17-340 m/s.

In yet another aspect, the disclosure provides methods and apparatus that employ a micropore array housed in a sorting cartridge. In one aspect, the sorting cartridge of the disclosure is designed so that the starting population of cells is never in physical contact with, or exposed to the surfaces of, the sorting instrument during use. In some aspects, the disclosure provides a reversibly closed system for sorting heterogeneous populations of cells to isolate a subpopulation with desirable characteristics. In some embodiments, the closed system comprises a cartridge defining an internal chamber, wherein the chamber is reversibly open to the external environment. In embodiments, the chamber of the cartridge houses a removable micropore array and collection tray. In some embodiments, the sorting cartridge of the disclosure comprises a humidity controlled cartridge with a humidification membrane placed on top of the array to reduce evaporation from the pores, and a humidification reservoir to provide a source of humidify within the cartridge. In a further aspect, the sorting cartridge is sterilized before use, and is disposed after use.

In exemplary embodiments, the cartridge of the disclosure is comprised of a transparent slide top cover slip wherein the cover is situated on top of an array mount with a wet cellulose membrane sandwiched between these two layers. The array mount of the disclosure defines an open, central region bordered on all sides by the array mount. The array mount further comprises a humidification reservoir connected to the open region. The array mount is situated on top of a transparent slide bottom cover. A removable collection tray is situated on the transparent slide bottom cover in the open region of the array mount. A micropore array is placed above the removable collection tray in the open region of the array mount. In this embodiment, the collection tray and top cover slip seals to form the barriers of the fully enclosed cartridge.

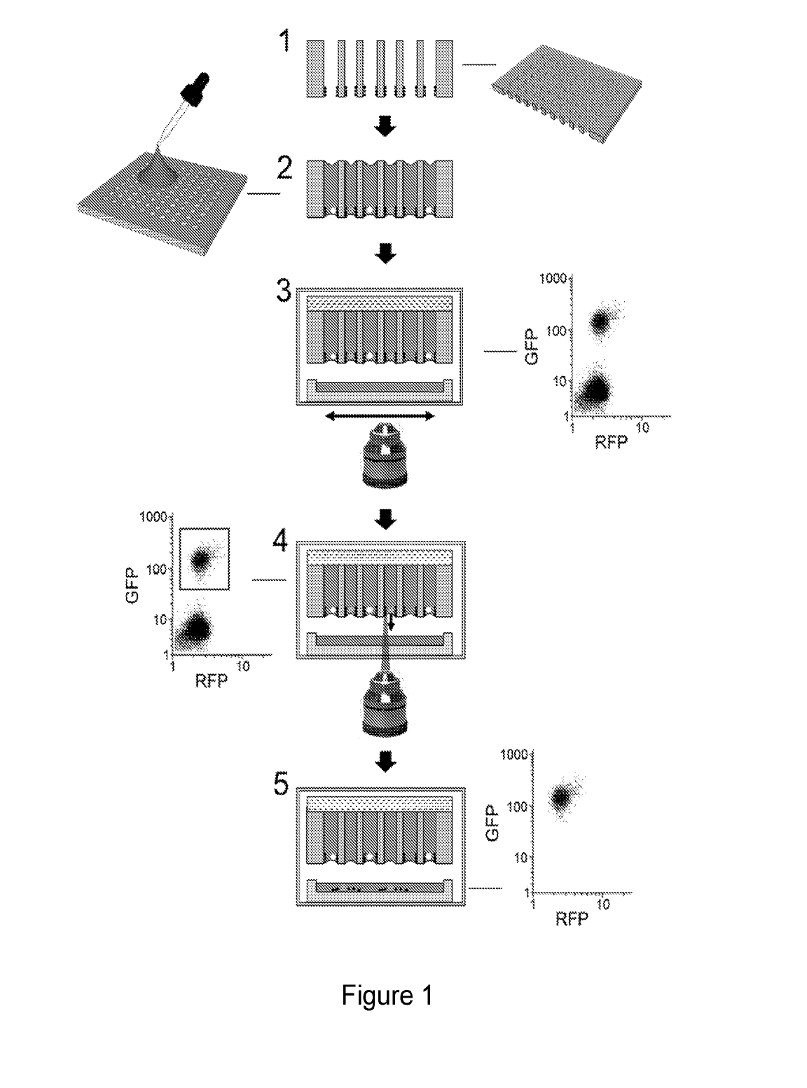

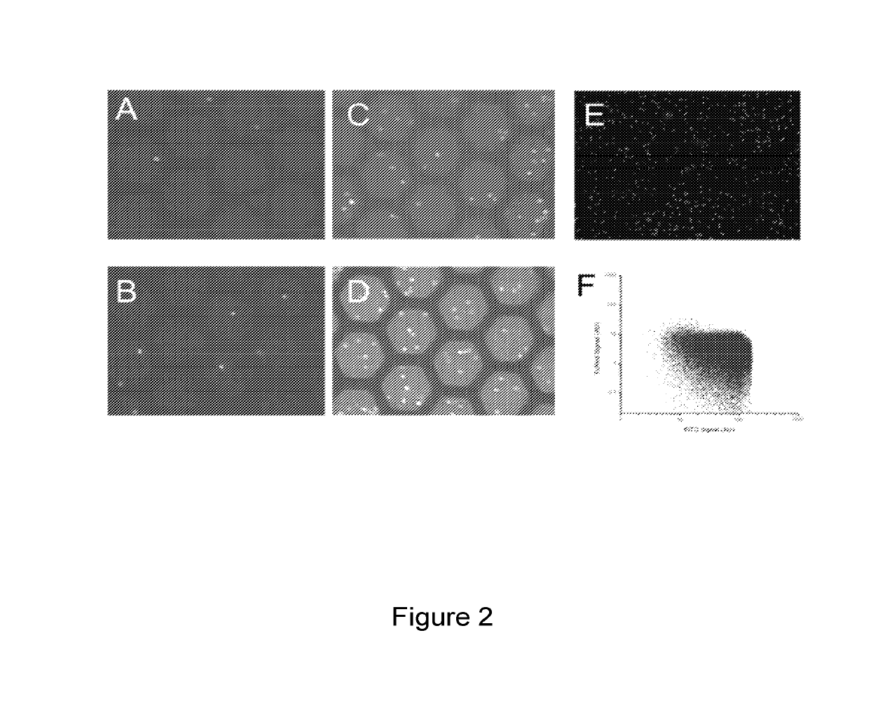

An example micropore clinical cell sorting principle is show FIG. 1. Step 1: An empty and disposable glass micropore array is used (shown in cross-section). The array includes of pores with a user defined diameter from 5 μm to 150 μm and a very high interlaced packing density (array open areas ˜67%). Thus for pores with a diameter of 15 μm a 10×10 inch plate contains approximately 240 million pores. The lower inner walls of each pore are coated with a layer of 2 um opaque polymer shell iron oxide microspheres (black). in Step 2: The micropore array is loaded with cells by placing the sample in contact with the array and spreading it along the array surface. The hydrophilic micropore array automatically absorbs and evenly meters the sample that enters each pore. Cells in the sample are Poisson randomly distributed across all the pores (See FIG. 2). Cells (dark for GFP+ and white for GFP−) settle at the bottom of each pore. Surface tension keeps the sample within the pore. In Step 3, the micropore array is loaded within a closed cartridge in which the humidity is controlled. In addition humidification membrane is placed on top of the array to reduce evaporation from the pores. A media loaded transparent collection tray is placed below the array with the closed cartridge. This tray will collect the sorted cells. Step 3: The closed cartridge is placed into an automated fluorescent scanning system that images the cells present in the micropore array. The image information is quantified and presented in a scatter plot where the signal from each cell is plotted. Step 4: Cells of interest are gated and extracted from the micropore array. Cells are extracted by locating the target pores and exposing them to a nanosecond laser pulse that is absorbed by the iron oxide microspheres. This causes a highly localized heating and expansion of the fluid on the surface of the microspheres. This expansion breaks the micropore surface tension and pushed the contents of the pore into the collection tray. Step 5: The collection tray is removed from the cartridge and sorted cells are gathered for downstream use, e.g., transplantation.

Microcavity Arrays

In embodiments, the arrays of the disclosure include cavities, or pores, included in an extreme-density porous array. See U.S. patent application Ser. No. 15/050,130. In an exemplary embodiment, the array is made from hydrophilic material (fused silica, “glass”), allowing the array to automatically absorb the sample, and resulting in random, Poisson distribution of cells into the pores.

In one example embodiment, a glass micropore array has an open array fraction of 66% and 100 μm pore diameter; an array width of 20 mm, length of 20 mm, and height of 1 mm. The pores are open on both ends (See FIG. 1). The sample and the cells are kept inside the pores by surface tension. The loaded micropore array creates a static array of the sample cells. If the number of cells is less than the number of loaded pores, then most pores will contain no cells or a single cell. In various aspects, the size of the arrays can range for a few millimeters to up to 100 centimeters.

In one embodiment, the surfaces of pores are coated with magnetic microparticles (Dynabeads® MyOne™ Streptavidin C1, Life Technologies). The coating may be accomplished by loading the particles into the array, sealing the bottom of the array with adhesive (e.g, APF-1, SDI Americas, Irvine Calif.) and then baking the array at, for example, 50° C. until the particles are adhered to the wall of the pores (e.g., 24 hours). The adhesive is then removed, leaving the particles adhered to the inside surfaces of each pore.

In other embodiments, each micro-pore can have a 5 μm diameter and approximately 66% open space (i.e., representing the lumen of each microcavity). In some arrays, the proportion of the array that is open ranges between about 50% and about 90%, for example about 60 to 75%, more particularly about 67%. In one example, a 10×10 cm array having 5 μm diameter microcavities and approximately 66% open space has about 330 million micro-pores. The internal diameter of micro-cavities may range between approximately 1.0 micrometers and 500 micrometers. In some arrays, each of the micro-pores can have an internal diameter in the range between approximately 1.0 micrometers and 300 micrometers; optionally between approximately 1.0 micrometers and 100 micrometers; further optionally between approximately 1.0 micrometers and 75 micrometers; still further optionally between approximately 1.0 micrometers and 50 micrometers, still further optionally, between approximately 5.0 micrometers and 50 micrometers.

In some arrays, the open area of the array comprises up to 90% of the open area (OA), so that, when the cavity size varies between 10 μm and 500 μm, the number of micro-pores per cm of the array varies between 458 and 1,146,500. In some arrays, the open area of the array comprises about 67% of the open area, so that, when the cavity size varies between 10 μm and 500 μm, the number of micro-pores per square cm of the array varies between 341 and 853,503. As an example, with a cavity size of 1 μm and up to 90% open area, each square cm of the array will accommodate up to approximately 11,466,000 micro-pores.

In one particular embodiment, a microcavity array can be manufactured by bonding billions of silica capillaries and then fusing them together through a thermal process. After that slices (0.5 mm or more) are cut out to form a very high aspect ratio glass micro perforated array plate. See, U.S. patent application Ser. No. 15/050,130. A number of useful arrays are commercially available, such as from Hamamatsu Photonics K. K. (Japan), Incom, Inc. (Massachusetts), Photonics Technologies, S.A.S. (France) Inc. and others. In some embodiments, the microcavities of the array are closed at one end with a solid substrate attached to the array.

The array may be loaded by contacting a solution containing a plurality of cells, such as a heterogeneous population of cells, with the array. In one embodiment, loading a mixture of a heterogeneous population of cells, e.g., mammalian bone marrow cells, peripheral hematopoietic cells, differentiated ES cells, or iPSCs cells, evenly into all the microcavities of a 20 mm×20 mm×1 mm array (approximately 1.4 million pores) involves placing a 150 μL droplet on the upper side of the array and spreading it over all the micro-pores. As an example, an initial concentration of cells results in an average distribution of approximately 1 cell per micro-cavity such that the number of pores with 2 or more cells is limited.

The volume of the cell-containing volume loaded onto the array will depend on several variables, including for example the desired application, the concentration of the heterogeneous mixture, and/or the desired dilution of the cells. In one specific embodiment, the desired volume on the array surface is about 0.25 to about 1 microliter per square millimeter. The concentration conditions are determined such that the cells are distributed in any desired pattern or dilution. In a specific embodiment, the concentration conditions are set such that in most cavities of the array only less than a single cell is present.

When the array is properly loaded, cells should randomly distribute into the array following Poisson distribution. According to this distribution, the probability, P, of loading a k number of cells in a microcapillary, where λ is the bulk concentration (average number of cells in the microcapillary volume), is calculated by the following equation:

$$P ( k , λ ) = \frac{{λ}^{k} {e}^{- λ}}{k !} \qquad ( Equation 1 )$$

For single-cell per microcapillary (k=1), the equation becomes:

P(1,λ)=λe^{-λ} (Equation 2)

Then to maximize the fraction of one cell per microcapillary, the local maximum of the equation 2 must be zero. Taking the derivative of equation 2:

P′^{(1,λ)}=e^{-λ}−λe^{-λ}=e^{-λ}(1−λ)=0

The concentration of the loading mixture is related to the average number of cells per microcapillary, 0.1, and microcapillary volume, (V_{capillary}) by the following equation:

$$Loading volume = \frac{λ}{{V}_{capillary}}$$

In other embodiments, the sample containing the heterogeneous population of cells may require preparation steps, e.g., incubation, after addition to the array. In other embodiments, each cell within each cavity is expanded (cells grown, phages multiplied, proteins expressed and released, etc.) during an incubation period.

A heterogeneous population of a single cell solution may first be debulked or enriched by density based separation or magnetic separation, however cell samples need not necessarily be so processed. After cells of interest have been loaded into the array, additional molecules or particles can be added or removed from the array without disturbing the cells. For example, any biological reactive molecule or particle useful in the detection of the cells can be added. These additional molecules or particles can be added to the array by introducing liquid reagents comprising the molecules or particles to the top of the array, such as for example by adding dropwise as described herein in relation to the addition of the cells.

Identification of Cavities Containing Cells of Interest

Following sample loading, addition of components, and/or another preparation step, the array is scanned to identify cavities containing cells having a phenotype of interest. For example, following established guidelines for quantitative wide-field microscopy, the intercapillary variability in fluorescence signals detected from the array may be measured. The passive nature of the microcapillary filling process results in a uniform meniscus level across the entire array. This uniformity, coupled with gravitational sedimentation of the loaded cells, simplifies the establishment of the imaging focus plane without the need for autofocus. Rather, the focus may be set at three distantly spaced points on the array, for example the corners. From these three points, the plane of the microcapillary array may be calculated.

Extraction of Microcavity Contents

Based on the optical information received from a detector associated with the array of cavities, target cavities containing biological elements, such as cells, with the desired properties are identified and their contents extracted. The disclosed methods maintain the integrity of the cells in the cavities. Therefore the methods disclosed herein provide for the display and independent recovery of a target population of biological cells from a population of up to billions of target cells. This is particularly advantageous for embodiments where cells are sorted.

For example, the signals from each cavity are scanned to locate the phenotypic characteristics of interest. This identifies the cavities of interest. Individual cavities containing the desired cells can be extracted using a method of extracting a solution including a biological element from a single microcavity in a microcavity array. In this embodiment, the microcavity is associated with an electromagnetic radiation absorbent material so that the material is within the cavity or is coating or covering the microcavity. Extraction occurs by focusing electromagnetic radiation at the microcavity to generate an expansion of the sample or of the material or both or evaporation that expels at least part of the sample from the microcavity. The electromagnetic radiation source may be the same or different than the source that excites a fluorescent label. The source may be capable of emitting multiple wavelengths of electromagnetic radiation in order to accommodate different absorption spectra of the materials and the labels.

In one aspect, extracting the contents of a pore of a micropore array comprises directing electromagnetic radiation at radiation absorbent material associated with the pores comprises a 15 nsec, 90 μJ laser pulse.

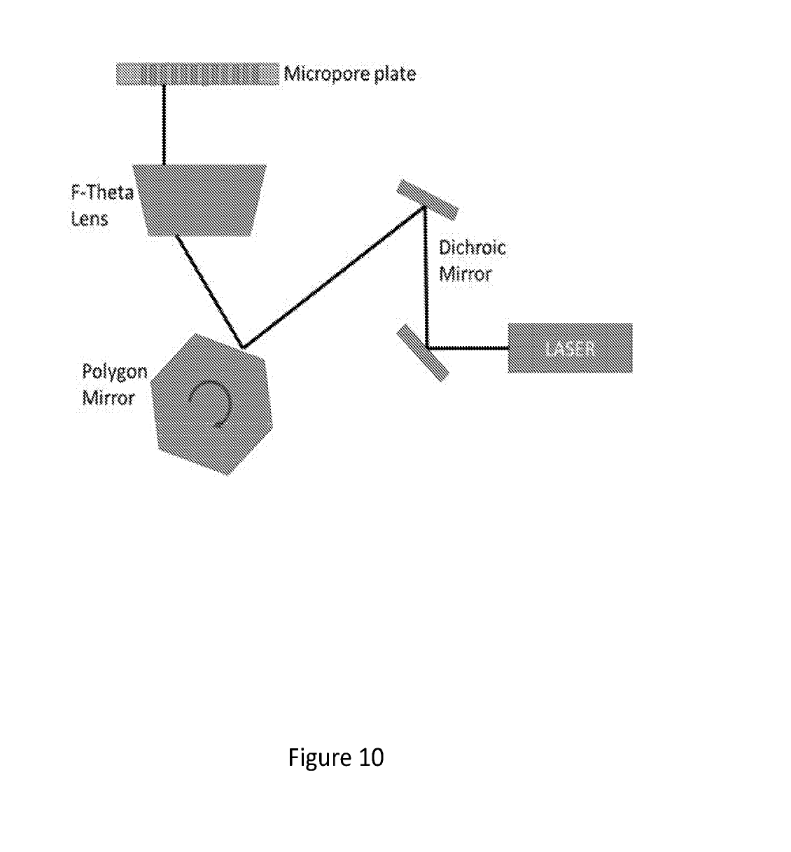

As on example of an embodiment of an ultrafast laser scanning system, FIG. 10 shows a laser scanning head comprised of a polygon mirror, F-theta optics and control electronics with phase locked-loop control (Lincoln Laser SOS-AB30) that can be used to for rapid extraction of the contents of the pore. With this system, scanning and/or extraction of the contents of the microcavities of each row of an array is associated with a facet of the mirror. Scanning of subsequent rows can be accomplished by moving the array or using second polygon mirror to move the laser focus to a subsequent row. A dichromatic mirror between the laser and the polygon mirror allows a signal from the plate to be captured by a detector (now shown). By rotating a mirror having 8 facets of 0.300″×1.810″ (Thickness×Diameter) size at a rate of 21,000-28,000 RPM, 10,000 microcavities with a pitch of 17 μm can be scanned in any single row in 0.003 seconds. Accordingly, an entire plate of 1.5 billion microcavities can be scanned in 450 seconds. In some embodiments, the scanning, imaging, and extracting steps according to the disclosure can sort between 2×10⁹ and 50×10⁹ cells within 120 minutes.

In some embodiments, subjecting a selected microcavity to focused electromagnetic radiation can cause an expansion of the electromagnetic radiation absorbent material, which expels sample contents onto a substrate for collecting the expelled contents.

In some embodiments the laser should have sufficient beam quality so that it can be focused to a spot size with a diameter roughly the same or smaller than the diameter of the pore. For instance, when the array material is capable of absorbing electromagnetic radiation, for instance when the array is manufactured or coated with an electromagnetic radiation absorbing material, the laser spot diameter may be smaller than the capillary diameter with the laser focused at the material-sample interface. In some embodiments, the material of the array itself, without any coating, such a darkened or blackened capillary array, can function as the electromagnetic radiation absorbent material. For example, as further described herein, array may be constructed of a lead glass that has been reduced in a hydrogen atmosphere. In various embodiments, the focus of the laser may be 90%, 80%, 70%, 60%, 50%, 40%, 30%, 20% 10%, 9%, 8%, 7%, 6%, 4%, 4%, 3%, 2% or 1% the diameter of the cavity.

In one aspect, the electromagnetic radiation is focused on the electromagnetic radiation absorbing material, resulting in linear absorption of the laser energy and cavitation of the liquid sample at the material/liquid interface. The electromagnetic radiation causes an intense localized heating of an electromagnetic radiation absorbing material of the array causing explosive vaporization and expansion of a thin layer of fluid in contact with the material without heating the remainder of the contents of the cavity. In most applications, directing of electromagnetic radiation to the material should avoid heating that liquid that is not in contact with the material at the focus of the radiation to avoid heating the liquid contents of the microcavity and impacting the biological material in the cells. Accordingly, while a very thin layer of liquid in proximity the focus of the electromagnetic radiation is heated to cause the explosive evaporation and expansion of the liquid, the amount of energy necessary to disrupt the meniscus is not sufficient to cause a significant increase in temperature of the entire liquid contents. In one aspect the laser is focused on the material of a cavity of the array adjacent the meniscus itself, causing a disruption of the meniscus without heating the liquid contents of the cavity other than the heating associated with the vaporization of a small amount of liquid at the portion of the meniscus adjacent the laser focus.

In certain embodiments, extraction from cavities of the array is accomplished by excitation of one or more particles in the microcavity, wherein excitation energy is focused on the particles. Accordingly, some embodiments employ energy absorbing particles in the cavities and an electromagnetic radiation source capable of discreetly delivering electromagnetic radiation to the particles in each cavity of the array. In certain embodiments energy is transferred to the particles with minimal or no increase in the temperature of the solution within the microcavity. In certain aspects, a sequence of pulses repeatedly agitates magnetic beads in a cavity to disrupt a meniscus, which expels sample contents onto a substrate for collecting the expelled contents.

In embodiments, the micopore array of the disclosure further comprises particles associated with the pores of the array. In certain embodiments, the particles associated with the pores of the array comprise magnetic particles, which may be adhered to the inner surfaces of the pores as described herein. In still further embodiments, the particles associated with the pores of the array comprise a radiation absorbent material. In particular embodiments, the particles associated with the pores of the array comprise iron oxide microspheres. In other embodiments, the particles associated with the pores of the array comprise opaque polymer shell iron oxide microspheres. In another particular embodiment, the particles associated with the pores of the array comprise Dynabeads®.

In certain embodiments, the particles associated with the pores of the array localize to the interior walls of the pores of the array. In some embodiments, the particles associated with the pores of the array coat the sides of the pores. In particular embodiments, the particles coat the sides of the pores of the array proximal to one open end of the pore. In alternative embodiments, the particles coat the sides of the array uniformly throughout the poor.

The electromagnetic radiation emission spectra from the electromagnetic radiation source must be such that there is at least a partial overlap in the absorption spectra of the electromagnetic radiation absorbent material associated with the cavity. In certain embodiments, individual cavities from a microcavity array are extracted by a sequence of short laser pulses rather than a single large pulse. For example, a laser is pulsed at wavelengths of between about 300 and 650, more particularly about 349 nm, 405 nm, 450 nm, or 635 nm. The peak power of the laser may be between, for example, approximately 50 mW and 100 mW. Also, the pulse length of the laser may be from about 1 msec to about 100 msec. In certain embodiments, the total pulse energy of the laser is between about 10 μJ and about 10 mJ, for instance 10, 25, 50, 100, 500, 1000, 2500, 5000, 7500, or 10,000 μJ. In certain embodiments, the diameter of the focus spot of the laser beam waist is between about 1 μm and about 20 [symbol] m, for instance 1, 2, 3, 4, 5, 6, 7, 8, 9, 10, 11, 12, 13, 14, 15, 16, 17, 18, 19 or 20 μm. In a particular example embodiment, the laser is pulsed at 75 mW peak power, 1 msec pulse length, 10 msec pulse separation, 2 μm diameter beam, with a total of 10 pulses per extraction.

In some embodiments, cavities of interest are selected and then extracted by focusing a 349 nm solid state UV laser at 20-30% intensity power. In one example, the source is a frequency tripled, pulsed solid-state Nd:YAG or Nd:YVO4 laser source emitting about 1 microJoule to about 1 milliJoule pulses in about a 50 nanosecond pulse. In another example, the source is a diode-pumped Q-switched Nd:YLF Triton UV 349 nm laser (Spectra-Physics). For instance, the laser may have a with a total operation time of about 15-25 ms, delivering a train of 35-55 pulses at about 2-3 kHz, at a pulse width of about 8-18 nsec, with a beam diameter of about 4-6 μm, and total energy output of 80-120 μJ. In one particular example, the laser may have a with a total operation time of about 15-20 ms, delivering a train of about 41-53 pulses at about 2.5 kHz, at a pulse width of about 10-15 nsec, with a beam diameter of about 5 μm, and total power output of 100 μJ. Both continuous wave lasers with a shutter and pulsed laser sources can be used in accordance with the disclosure.

In some embodiments, a diode laser may be used as an electromagnetic radiation source. In certain embodiments, the focus of diode laser has a beam waist diameter between about 1 [symbol] m and about 10 [symbol] m, for instance a 1, 2, 3, 4, 5, 6, 7, 8, 9 or 10 μm diameter. The diode laser may have a peak power of between about 20 mW and about 200 mW peak power, for instance about 20 mW, 40 mW, 60 mW, 80 mW, 100 mW, 110 mW, 120 mW, 130 mW, 140 mW, 150 mW, 160 mW, 170 mW, 180 mW, 190 mW or 200 mW peak power. The diode laser can be used at wavelengths of between about 300 and about 2000 nm, for instance about 405 nm, 450 nm, or 635 nm wavelength. In other embodiments, an infrared diode laser is used at about 800 nm, 980 nm, 1300 nm, 1550 nm, or 2000 nm wavelengths. Longer wavelengths are expected to have less photoxicity for any given sample.

In certain embodiments, a diode laser is pulsed at between about 2 to 20 pulses, for instance 2, 4, 6, 8, 10, 12, 14, 16, 18, and 20 pulses, with a pulse length of about 1 to 10 msec, for instance, 1, 2, 3, 4, 5, 6, 7, 8, 9, and 10 msec, and having a pulse separation of approximately 10 msec to 100 msec, for instance 10, 20, 30, 40, 50, 60, 70, 80, 90 and 100 msec. In an example embodiment, the diode laser is an Oclaro HL63133DG laser with a peak power of 170 mW operating at a wavelength of 635 nm. In another example embodiment, the diode laser is an Osram PL450B laser operating at 450 nm.

In other example embodiments, a diode laser or a Triton laser are focused to diameters of between 1 to 10 microns. The lasers emit a train of 10 to 50 pulses over a time period of 10 msec to 100 msec. Each individual pulse has a time duration of 1 msec (diode laser) or 10 nsec (Triton laser). The total pulse train energy is approximately 100 microJoules. The laser energy is absorbed within a volume in the microcapillary which is approximately a cylinder with a diameter roughly equal to the diameter of the laser beam waist and a height determined by the absorption length of the laser beam. If magnetic beads are in the capillary the laser pulse energy is absorbed by the beads, primarily heating the surface of the bead that is directly exposed to the laser. The liquid in immediate proximity to this surface is explosively vaporized which propels the beads within the capillary. The explosive motion of the beads along with vaporization of the nearby liquid disrupts the meniscus and empties the capillary. If the material of the array itself absorbs the light then the laser energy is deposited primarily in the portion of the capillary wall upon which the laser is incident. If sufficient laser energy is absorbed in this absorbing volume in a short enough time, then the heat will not have time to diffuse to the surrounding liquid. The liquid in the absorption volume will be explosively vaporized by the laser pulse, causing a rapid expansion of a portion of the sample, which disrupts the meniscus and empties the contents of the microcapillary, and heat diffusion to the surrounding liquid outside of the absorbing volume will be minimized.

In a particular example, an individual laser pulse has a duration of approximately 1 msec and the beam waist diameter is approximately 10 microns. In this example, the single laser pulse will heat the volume of liquid within the absorption region of the laser beam and during the pulse the heat will diffuse only a few microns outside of the absorbing region. The energy deposited during the laser pulse causes the temperature of the liquid in the absorbing region to rise abruptly to many times the vaporization temperature. The liquid is explosively vaporized in this absorption region while the surrounding region stays essentially at its original temperature. The explosive vaporization of liquid within the absorbing region disrupts the meniscus and the liquid is expelled from the microcapillary with negligible heat diffusion from the absorbent material to the surrounding medium and resulting in negligible or no heating of the total liquid contents of the microcapillary.

The equation describing the distance of propagation of heat within a substance over a short time scale is:

(d=\sqrt{(α*τ)}).

Where d is the characteristic thermal diffusion distance, α is the thermal diffusion coefficient, and τ is the energy deposition time or laser pulse length. For water α=0.143 mm²/sec and with T=1 msec this equation results in a predicted diffusion length of about 10 microns. A total pulse energy of 100 microJoules deposited in the approximate absorption cylinder volume determine by a beam with a waist diameter of 10 microns and a height of 10 microns (˜10e-12 cm³) will raise the temperature of the liquid in this volume to many, many times the evaporation temperature of the liquid, resulting in explosive expansion of liquid in this volume.

The Veritas laser supplies a train of about 40, 5 nsec pulses, each pulse separated by about 500 microseconds. Each pulse causes explosive expansion of the liquid in the absorbing volume, propelling the beads (if present) and disrupting the meniscus. The diode laser similarly delivers a train of ten 1 msec pulses separated by several milliseconds, which interacts with liquid in the capillary in a similar fashion. In both cases using multiple pulses in a pulse train enhances the extraction efficiency compared to using a single high energy pulse.

When microspheres used, the equation for the thermal relaxation time ( ) for uniform spheres of diameter d is

$${t}_{r} = \frac{{d}^{2}}{27 k} = \frac{{( 1 μm )}^{2}}{27 * .143 \times {10}^{- 6} \frac{{m}^{2}}{s}} = 259 ns$$

As long as the laser pulse is <˜300 ns (this changes depending on the diameter of the beads), there will be thermal confinement and rapid localized heating of the absorbent material.

In further example embodiments, the following parameters may be used

### 1) Laser Parameters

a. Veritas laser
   i. Triton UV 349 nm laser (diode-pumped Q-switched Nd:YLF laser, Spectra-Physics)
   ii. Total operation time: 18±2 ms (n=5 measurements), delivering a train of 46.6±5.9 pulses at 2.5 kHz
   iii. Pulse width: 10-15 nsec
   iv. Beam diameter: 5 μm
   v. Total power: 100 μJ
      2) Absorbing material
a. Superparamagnetic iron oxide-doped microbeads
   i. Diameter ˜1 um (can range from 100 nm-10 um)
   Thermal relaxation time:

$${t}_{r} = \frac{{d}^{2}}{27 k} = \frac{{( 1 μm )}^{2}}{27 * .143 \times {10}^{- 6} \frac{{m}^{2}}{s}} = 259 ns$$

b. Black capillary walls (e.g., lead-silicate layer from reducing alkaline-doped silicate glass in a hydrogen atmosphere).

Materials within the cavity can be, for example, the particles described above. In addition to directly adhering the particles to the surfaces of the cavities as described herein, these particles may be functionalized so that they bind to the walls of the micro-cavities. Similar materials can be used to coat or cover the microcavities, and in particular, high expansion materials, such as EXPANCEL® coatings (AkzoNobel, Sweden). In another embodiment the EXPANCEL® material can be supplied in the form of an adhesive layer that is bonded to one side of the array so that each cavity is bonded to an expansion layer.

Focusing electromagnetic radiation at a microcavity can cause the electromagnetic radiation absorbing material to expand, which causes at least part of the liquid volume of the cavity to be expelled. When the material is heated to cause rapid expansion of the cavity content, a portion of the of the contents may be expanded up to, for example, 1600 times, which causes a portion of the remainder of the contents to be expelled from the cavity.

Collection of Extracted Cells

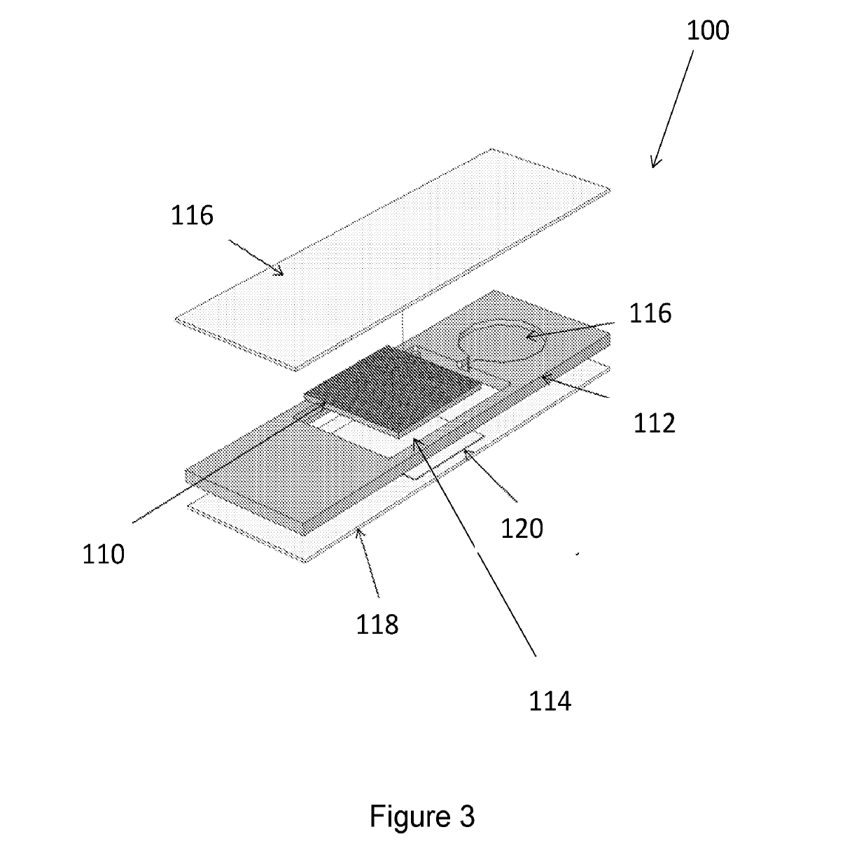

In order to collect the content of cavities identified as containing cells having a phenotype of interest, a sorting cartridge is provided. The cartridge may be machined using laser ablation and lamination. The cartridge prevents the collected contents of the array from contacting surfaces within the sorting instrument, thus preventing cross-contamination between samples. The cartridge is also sealed to avoid evaporation of media and aerosolization of cells. The cartridge may have a transparent slide top and bottom covers for imaging and laser extraction and a cell capture tray for collecting the sorted cells. As shown in FIG. 3, the cartridge 100 includes the micropore array 110 mounted in an array mount 112 having an opening 114 that is suitably sized for the array 110. A top transparent cover 116 and a bottom transparent cover 118 seals the array 110 in the opening 114. The array mount can include a humidification reservoir 116 containing a humidification fluid (not shown) that helps to prevent the fluid in the cavities of the array from drying out. A removable transparent collection tray 120 is located between the bottom cover.

Electromagnetic Radiation Source

In one embodiment, the electromagnetic radiation source of the apparatus is broad spectrum light or a monochromatic light source having a wavelength that matches the wavelength of at least one label in a sample. In a further embodiment, the electromagnetic radiation source is a laser, such as a continuous wave laser. In yet a further embodiment, the electromagnetic source is a solid state UV laser. A non-limiting list of other suitable electromagnetic radiation sources include: argon lasers, krypton, helium-neon, helium-cadmium types, and diode lasers. In some embodiments, the electromagnetic source is one or more continuous wave lasers, arc lamps, or LEDs.

In some embodiments, the apparatus comprises multiple (one or more) electromagnetic sources. In other embodiments, the multiple electromagnetic (EM) radiation sources emit electromagnetic radiation at the same wavelengths. In other embodiments, the multiple electromagnetic sources emit different wavelengths in order to accommodate the different absorption spectra of the various labels that may be in the sample.

In some embodiments, the multiple electromagnetic radiation sources comprise a Triton UV laser (diode-pumped Q-switched Nd:YLF laser, Spectra-Physics) operating at a wavelength of 349 nm, a focused beam diameter of 5 μm, and a pulse duration of 20 ns. In still further embodiments, the multiple electromagnetic radiation sources comprise an X-cite 120 illumination system (EXFO Photonic Solutions Inc.) with a XF410 QMAX FITC and a XF406 QMAX red filter set (Omega Optical). In an example embodiment, a diode laser is a Oclaro HL63133DG laser with a peak power of 170 mW operating at a wavelength of 635 nm. In another example embodiment, the diode laser is an Osram PL450B laser operating at 450 nm.

The apparatus also includes a detector that receives electromagnetic (EM) radiation from the label(s) in the sample, array. The detectors can identify at least one cavity (e.g., a microcavity) emitting electromagnetic radiation from one or more labels.

In one embodiment, light (e.g., light in the ultra-violet, visible or infrared range) emitted by a fluorescent label after exposure to electromagnetic radiation is detected. The detector or detectors are capable of capturing the amplitude and duration of photon bursts from a fluorescent moiety, and further converting the amplitude and duration of the photon burst to electrical signals. In some embodiments the detector or detectors are inverted.

Once a cell is labeled to render it detectable, or if the cell possesses an intrinsic characteristic rendering it detectable, any suitable detection mechanism known in the art may be used without departing from the scope of the disclosure, for example a CCD camera, a video input module camera, a Streak camera, a bolometer, a photodiode, a photodiode array, avalanche photodiodes, and photomultipliers producing sequential signals, and combinations thereof. Different characteristics of the electromagnetic radiation may be detected including: emission wavelength, emission intensity, burst size, burst duration, fluorescence polarization, and any combination thereof. As one example, a detector compatible with the disclosure is an inverted fluorescence microscope with a 20× Plan Fluorite objective (numerical aperture: 0.45, CFI, WD: 7.4, Nikon) and an ORCA-ER cooled CCD camera (Hamamatsu).

The detection process can also be automated, wherein the apparatus comprises an automated detector, such as a laser scanning microscope.

In some embodiments, the apparatus as disclosed can comprise at least one detector; in other embodiments, the apparatus can comprise at least two detectors, and each detector can be chosen and configured to detect light energy at the specific wavelength range emitted by a label. For example, two separate detectors can be used to detect cells with different labels, which upon excitation with an electromagnetic source, will emit photons with energy in different spectra. In still further embodiments, the apparatus as disclosed can comprise more than two detectors, for example three, four, five, six, seven, eight, nine, or ten detectors.

Kits

In various aspects, the disclosure is directed to kits for the detection, identification, isolation and/or purification of populations of cells of interest. In some embodiments, the kits contain reagents capable of detecting phenotypic markers characteristic of a cell type of interest. In some embodiments, the kits of the disclosure comprise antibodies specific for phenotypic marker, such as a protein of interest, in addition to detection reagents and buffers. In various embodiments, the kits contain all of the components necessary to perform a identification of cells, including all controls, instructions for performing assays, and any necessary software for analysis. Labels may be conjugated to a binding partner for the phenotypic marker, or other sample component. The kits may also include second, third, or fourth, etc. set of reagents that are functionalized to bind second, third, or fourth, etc. phenotypic markers in cells of the sample. Stabilizers (e.g., antioxidants) to prevent degradation of the reagents by light or other adverse conditions may also be part of the kits. The kits may further comprise a reversibly closed, humidity controlled cartridge housing a micropore array and a removable collection tray, wherein the cartridge is capable of receiving a sample of heterogeneous cells processed using components of the kit.

While the instructional materials typically include written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD-ROM), and the like. Such media may include addresses to internet sites that provide such instructional materials.

In various aspects of the methods disclosed herein, the phenotype of interest is a cell surface binding agent, such as a protein expressed on the surface of a cell. In another aspect, the phenotype of interest is the expression of a protein in the interior of the cell. In another aspect, the phenotype of interest is the expression of a protein secreted from the cells. In yet another aspect, the phenotype of interest may be the production of a protein having enzymatic activity, a protein that modulates enzyme activity, wherein the enzyme activity is measurable according to the present methods, or creates a product measurable according to the present methods.

The method for identifying, isolating, sorting, and/or purifying a cell or population of cells from a heterogeneous population of cells disclosed herein allows for the simultaneous identification of two or more different phenotypic markers per pore. Therefore, in some embodiments, simultaneous positive and negative screening can occur in the same pore. This screening design improves the selectivity of sorting.

Antibodies

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various forms of the molecule, if present, or for total (e.g., all, or substantially all, of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments that mimic antibodies can be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest Ltd., Turk, Finland; Abcam Inc., Cambridge, Mass., USA, Life Diagnostics, Inc., West Chester, Pa., USA; Fitzgerald Industries International, Inc., Concord, Mass., USA; BiosPacific, Emeryville, Calif.).

In some embodiments, the antibody is a polyclonal antibody. In other embodiments, the antibody is a monoclonal antibody. In embodiments, the antibodies of the disclosure are compatible with downstream applications of the cell populations extracted according to the present methods. For example, the antibodies of the disclosure may be non-immunogenic, humanized antibodies. In some embodiments, the antibodies of the disclosure comprise an epitope tag useful to immobilize the antibody before or after extraction of the sample, thereby depleting the antibody from the extracted cell population.

Capture binding partners and detection binding partner pairs, e.g., capture and detection antibody pairs, can be used in embodiments of the disclosure. Thus, in some embodiments, a sorting and purification protocol is used in which, typically, two binding partners, e.g., two antibodies, are used. One binding partner is a capture partner, usually immobilized on a particle, and the other binding partner is a detection binding partner, typically with a detectable label attached. Such antibody pairs are available from several commercial sources, such as BiosPacific, Emeryville, Calif. Antibody pairs can also be designed and prepared by methods well-known in the art. In a particular embodiment, the antibody is biotinylated or biotin labelled

In one embodiment, there is a second imaging component that binds all members of the starting cell population non-specifically. Therefore, this signal can be read to normalize the quantity of fluorescence between cavities. One example is an antibody that will bind a protein or proteins that are ubiquitously expressed on the cell surface of a starting population of cells.

Labels

Several strategies that can be used for labeling binding partners to enable their detection or discrimination in a mixture of particles are well known in the art. The labels may be attached by any known means, including methods that utilize non-specific or specific interactions. In addition, labeling can be accomplished directly or through binding partners.

Emission, e.g., fluorescence, from the moiety should be sufficient to allow detection using the detectors as described herein. Generally, the compositions and methods of the disclosure utilize highly fluorescent moieties, e.g., a moiety capable of emitting electromagnetic radiation when stimulated by an electromagnetic radiation source at the excitation wavelength of the moiety. Several moieties are suitable for the compositions and methods of the disclosure.

Labels activatable by energy other than electromagnetic radiation are also useful in the disclosure. Such labels can be activated by, for example, electricity, heat or chemical reaction (e.g., chemiluminescent labels). Also, a number of enzymatically activated labels are well known to those in the art.

Typically, the fluorescence of the moiety involves a combination of quantum efficiency and lack of photobleaching sufficient that the moiety is detectable above background levels in the disclosed detectors, with the consistency necessary for the desired limit of detection, accuracy, and precision of the assay.

Furthermore, the moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems of the disclosure (e.g., does not cause precipitation of the protein of interest, or precipitation of a protein to which the moiety has been attached).

A fluorescent moiety may comprise a single entity (a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

In some embodiments, the fluorescent dye molecules comprise at least one substituted indolium ring system in which the substituent on the 3-carbon of the indolium ring contains a chemically reactive group or a conjugated substance. Examples include Alexa Fluor molecules.

In some embodiments, the labels comprise a first type and a second type of label, such as two different ALEXA FLUOR® dyes (Invitrogen), where the first type and second type of dye molecules have different emission spectra.

A non-inclusive list of useful fluorescent entities for use in the fluorescent moieties includes: ALEXA FLUOR® 488, ALEXA FLUOR® 532, ALEXA FLUOR® 555, ALEXA FLUOR® 647, ALEXA FLUOR® 700, ALEXA FLUOR® 750, Fluorescein, B-phycoerythrin, allophycocyanin, PBXL-3, Atto 590 and Qdot 605.

Labels may be attached to the particles or binding partners by any method known in the art, including, absorption, covalent binding, biotin/streptavidin or other binding pairs. In addition, the label may be attached through a linker. In some embodiments, the label is cleaved by the analyte, thereby releasing the label from the particle. Alternatively, the analyte may prevent cleavage of the linker.

### EXAMPLES

### Example 1: Micropore Arrays for Microarray Based Sorting

Microarray cell loading is performed by spreading the cells over the array with a pipette. A total of 150 μL is loaded into such an array. Generally, the arrays are loaded with an average of 0.5 cells per pore or less, such that the number of pores with 2 or more cells is limited. The number of cells per well is controlled by the concentration of cells in the starting solution.

A Veritas 704 Laser Microdissection Instrument (Arcturus Bioscience Inc.) was modified by changing the stage holder, the camera (to a Hamamatsu Orca-Er), and removing the cap-placing arm. Control software was written in Matlab R2010b to communicate with the Veritas machine via a serial port and control all aspects of the machine (X-Y stage, Z focus, extraction laser, fluorescent imaging, camera exposure, etc.). The software automatically scans the array and quantifies the fluorescence from each cell and generate scatter plots. Manually drawn gates on the scatter plots defined the target population. FIG. 2, Panel A-D shows fluorescent microscope images of cells loaded at 0.5(A), 1(B), 5 (C) and 10 (D) cells per pore into a 100 μm diameter micropore array. FIG. 2, Panel E shows a representative raw fluorescent image of a 20 μm diameter pore array loaded with GFP expressing cells (punctate staining pattern). FIG. 2, Panel F shows a scatter plot after automatically scanning and analyzing 0.5 million pores from the array in Panel E.

The software then automatically aimed the laser at each target pore to extract the contained cell. If the software detected two or more cells per pore, that pore was ignored and not extracted.

The extracted cells were captured in the media-containing collection tray located below the micropore array. The extraction of each cell occurred in less than 1.2 msec (high-speed camera data not shown), and probably in the order of tens of microseconds.

### Example 2: Confirmation of Cells from Single Cavity of Micropore Array

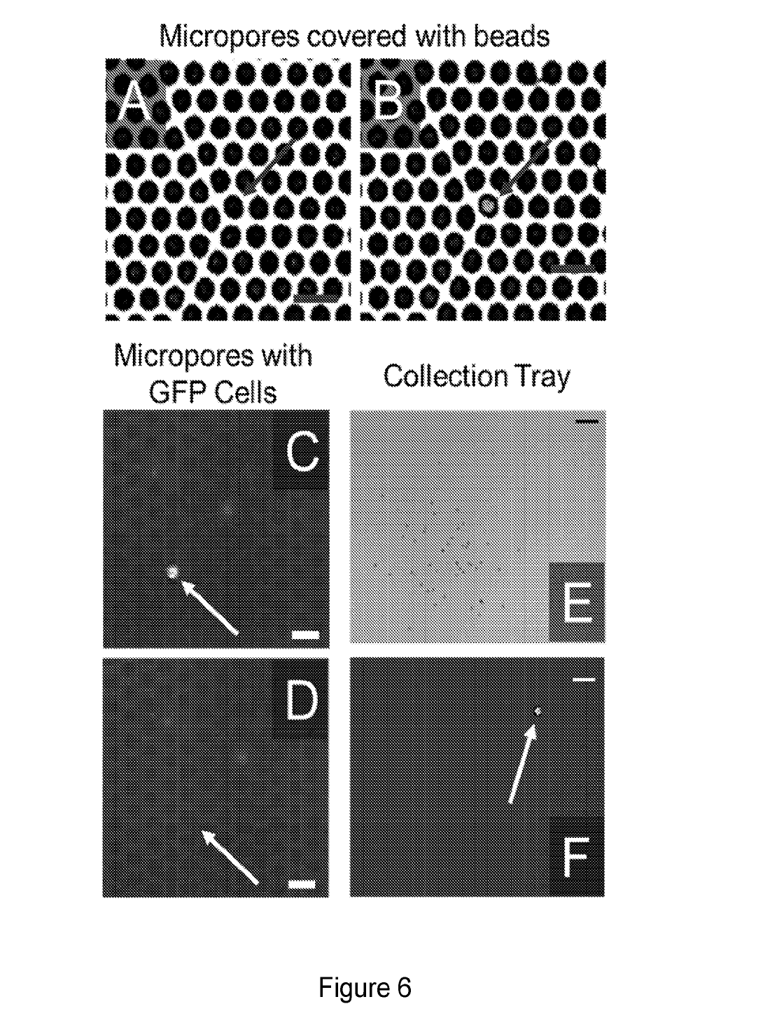

Extraction of a cell from a single well of the micropore array was demonstrated. The micropore array was filled with 2.8 μm magnetic particles and seeded with a population of GFP MOLM-13 cells. FIG. 6, Panel A shows a bright field image of the micropore array filled with 2.8 μm magnetic particles before the laser extraction. Panel B shows target pore after the laser pulse. The magnetic beads are extracted from the target pore and none of the neighboring pores were affected by the extraction. The micropore array was also imaged to detect GFP fluorescence signal emanated from cells in pores of the array. Panel C shows a GFP MOLM-13 cell targeted for extraction. Panel D shows the same site as after the laser extraction (as shown by the arrow) showing the absence of the target cell. (E-F) Finally, the collection tray was imaged by both bright-field and GFP fluorescence microscopy after extraction, showing the extraction of particles (Panel E) and the GFP positive cell (Panel F). All scale bars represent 40 μm.

### Example 3: Equivalent Cytometry Between Micropore Array Vs. FACS

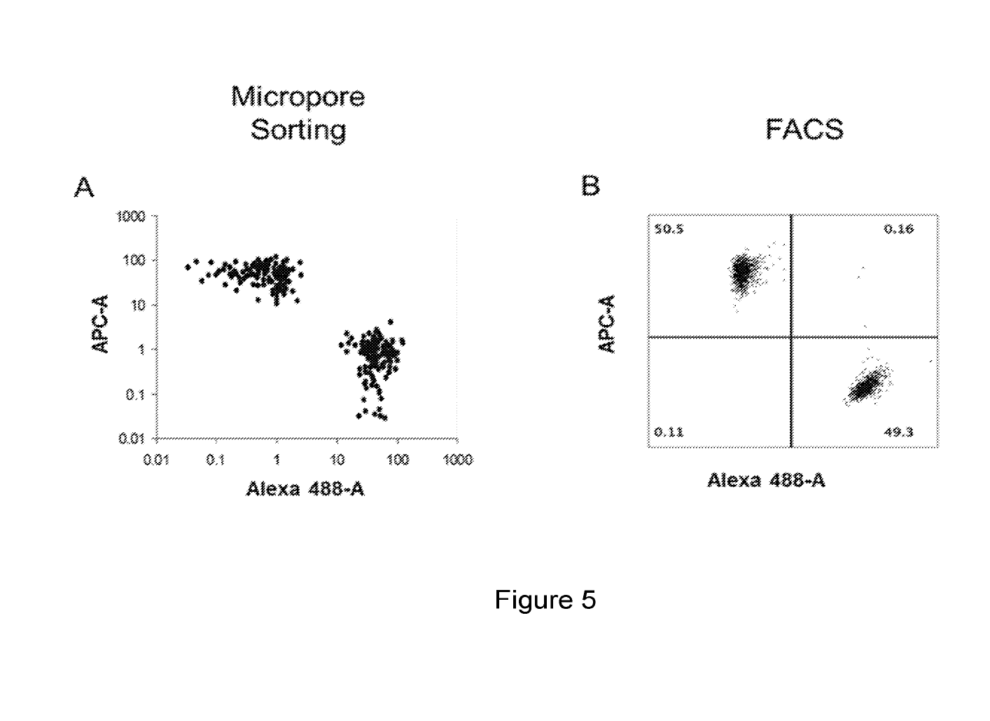

To determine the equivalence of cytometry detection between these two techniques, independent populations human bone marrow cells were first stained with either Carboxyfluorescein succinimidyl ester (CFSE) or CellTrac™ Far Red Cell. Cells were then mixed in equal proportion and matched samples were either loaded onto micropore arrays and analyzed or loaded onto a conventional flow cytometer and analyzed. After computational transformation of the data, equivalent analysis was obtained from both the micropore array and the flow cytometer in that both in terms of accuracy of properly counting events and the overall separation between the two populations (FIG. 5).

### Example 4: Viability and Functionality of Cells Sorted with Micropore Array

To demonstrate in vitro adherent cell culture viability, DLD-1 human colon cancer cells were cultured in DMEM (Gibco Invitrogen, Carlsbad, Calif., USA) supplemented with 10% fetal calf serum (FCS; Omega Scientific Inc., Tarzana, Calif., USA), 2 mM L-glutamine (Gibco Invitrogen), and 1× Penicillin-Streptomycin (Gibco Invitrogen). The cell lines were maintained in 25 cm² canted-neck flasks (BD Falcon, BD Biosciences, San Diego, Calif., USA) in 5% CO₂ at 37° C. and were split every three days to 1×10⁵ cells/mL.

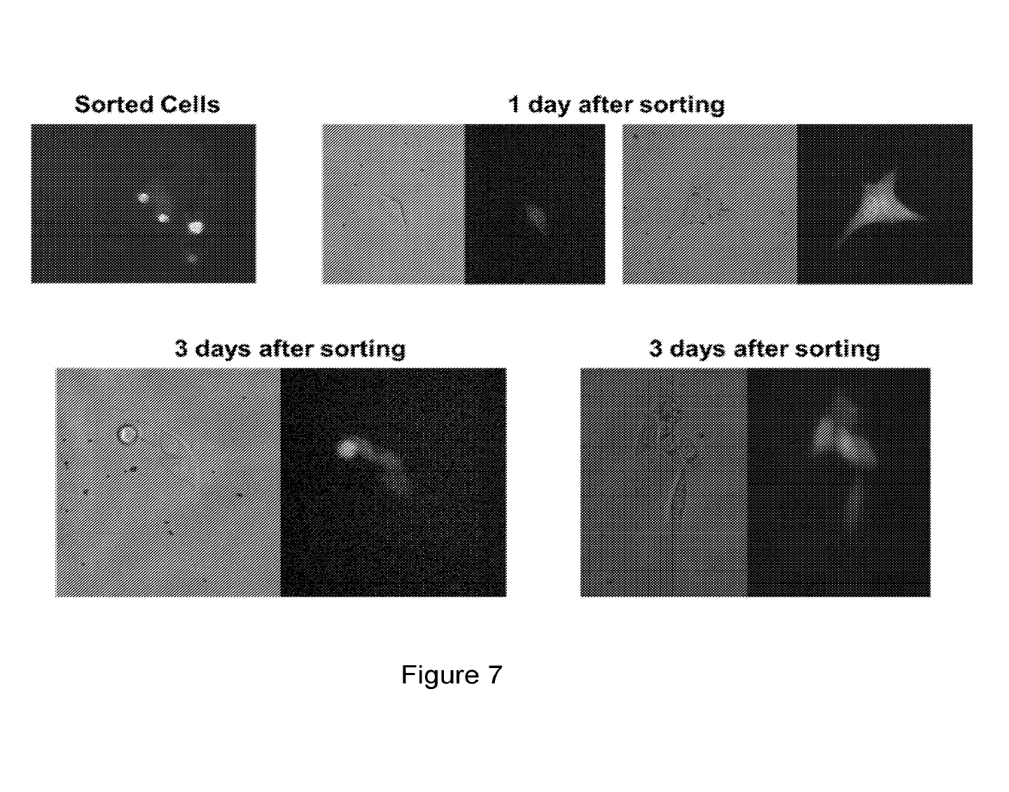

To address is whether the extracted cells are viable, were loaded into a micropore array and then laser extracted. The extracted cells were stained with propidium iodide. Cell viabilities were in the range of 70%-90% as assessed by fluorescent microscopy. The cells were then cultured as above and monitored the cells for adhesion by cell morphology and and growth at 72 hours (FIG. 7). No differences were noted between extracted and non-extracted cells.

### Example 5: In Vitro HSC Colony Formation of Cells Sorted with Micropore Array

In this Example, sorted HSCs were cultured in an alpha-Modified Eagle Medium (MEMα)-based methyl-cellulose media (Methocult M3100; StemCell Technologies, Vancouver, Canada) that was supplemented with 30% fetal bovine serum (FBS), 1% bovine serum albumin, 2 mM L-glutamine, and 50 μM 2-mercaptoethanol. Cytokines such as mouse SCF (100 ng/ml; provided by Immunex), mouse thrombopoietin (50 ng/ml), mouse IL-3 (30 ng/ml; Genzyme, Cambridge, Mass.), mouse IL-6 (10 ng/ml; Genzyme), mouse GM-CSF (10 ng/ml; Genzyme), M-CSF (25 U/ml; Genzyme), and erythropoietin (1 U/ml) were added at the start of the culture.

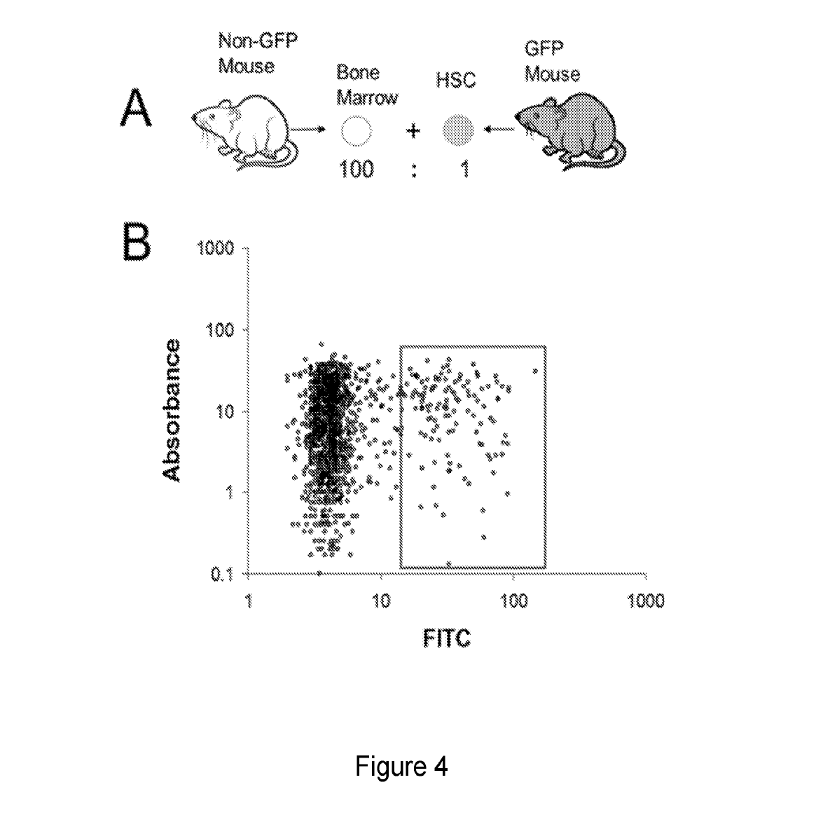

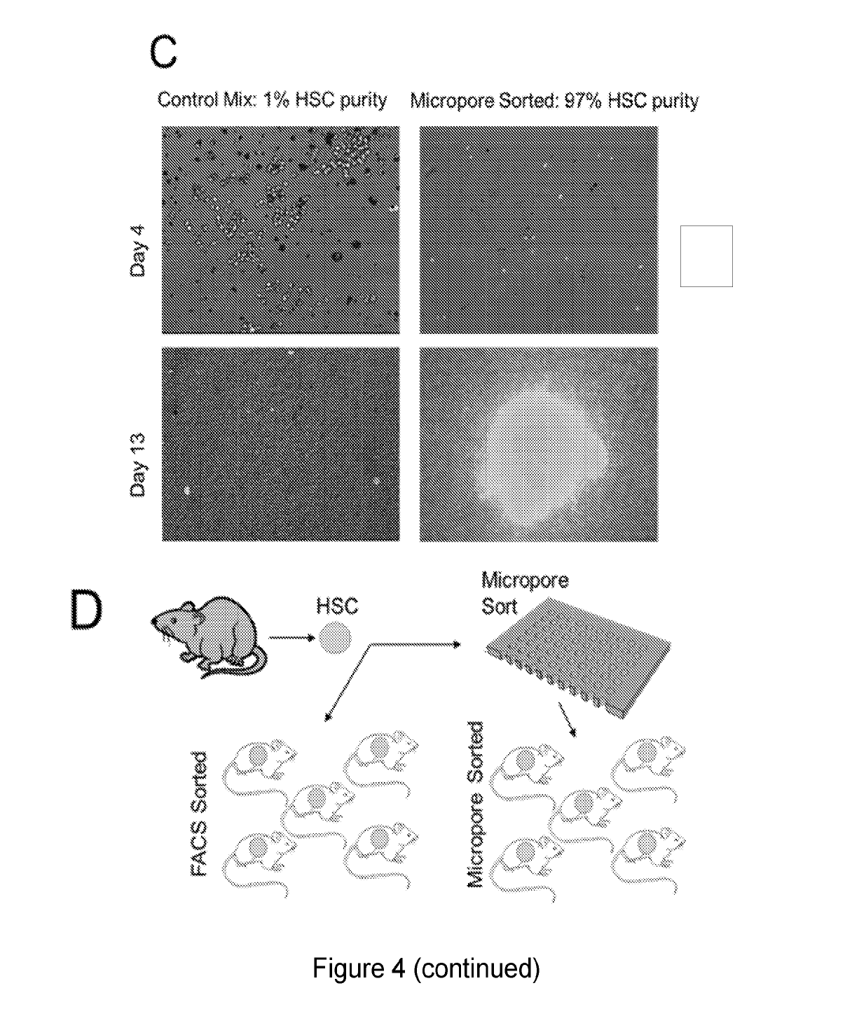

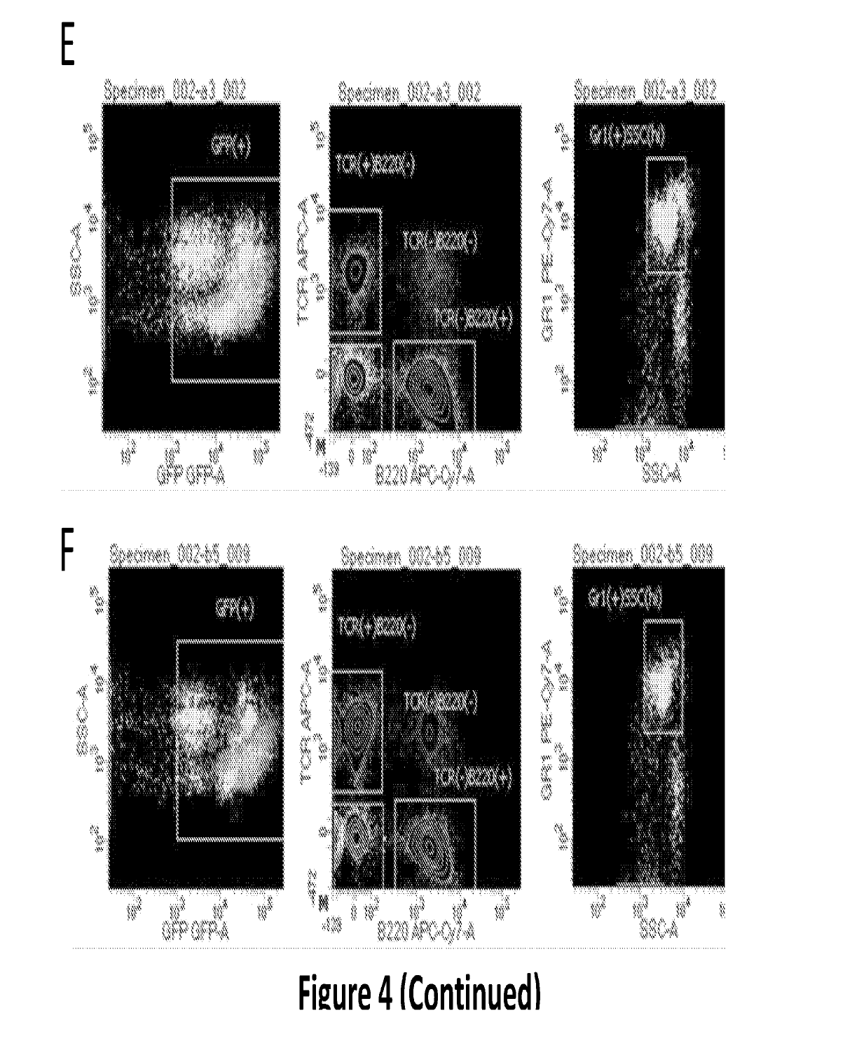

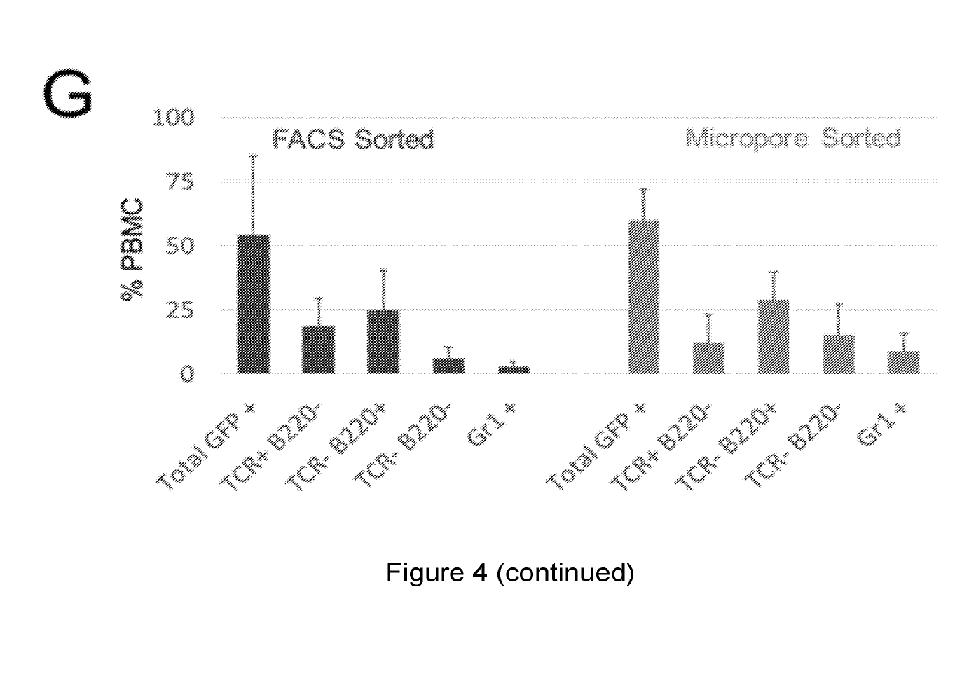

To evaluate viability of HSCs after sorting, primary mouse HSCs were extracted from bone marrow from a green-fluorescent-protein (GFP) mouse and purified using FACS. The pure GFP-HSC population was mixed with non-GFP mouse bone marrow cells (1:100 ratio) and loaded into the micropore array. (See FIG. 4, Panel A). Micropore-based cell sorting was used to extract the HSCs and cultured in methylcellulose HSC media. The extracted HSCs (97% purity) were cultured for over 14 days to form large GFP+ colonies. In contrast the unsorted control sample that contained GFP+ HSCs and GFP− bone marrow cells generated cell populations with weak GFP signals.

FIG. 4, Panel B shows a scatterplot from the micropore sorted GFP HSCs, and non-GFP bone marrow cells. The gated (box) population of GFP cells was isolated in a collection tray according to the disclosure and used for in vitro cultures. FIG. 4, Panel C shows in-vitro culture of the extracted HSCs (the population sorted in FIG. 4, Panel B) and the starting mixture (control).

### Example 6: In Vivo HSC Viability and Functionality of Cells Sorted with Micropore Array

Long term in-vivo functionality is a key indicator of the true health of cells. To verify whether the micropore sorted, laser extracted cells retain in-vivo viability and functionality, micropore sorted mouse GFP-HSCs are transplanted into non-GFP, lethally irradiated host-mice. FACS sorted GFP-HSCs are used as a parallel control. The long-term cell engraftment and blood forming capabilities of the sorted cells were determined. The GFP-HSCs sorted through the micropore laser based system successfully engrafted long-term and generated effector blood cells in the myeloid and lymphoid lineages. Micrographs show the cells at day 4 and 13 after extraction. FIG. 4, Panel D shows a schematic of the experiment to evaluate long-term in vivo functionality of HSCs sorted with the micropore array method. FIG. 4, Panels E and F show long-term engraftment analysis (18 weeks after HSC injection). FIG. 4 Panel E shows a representative distribution of GFP cells in mouse PBMCs for a mouse that received micropore re-sorted GFP-HSCs. FIG. 4, Panel F shows a representative distribution of GFP cells in mouse PBMCs for a mouse that received FACS sorted GFP-HSCs. FIG. 4, Panel G shows quantitation of the results presented Panels 4E and 4F. Direct comparison between FACS and micropore re-sorted GFP-HSCs showed that there is no statistical difference in the cell engraftment and blood forming capabilities (FIG. 4, Panel G). This indicated that HSC viability was retained and demonstrated the sorting and purification capabilities of the micropore based cell sorting concept (enriching the GFP-HSC purity from 1% to 97%). All the lethally irradiated mice that received the laser sorted HSCs survived for more than 18 weeks.

### Example 7: Composition of Sculpted Grafts for Production with Micropore Array

Micropore based sorting according to the disclosure enables construction of sculpted grafts of an appropriate size for transplantation in human patients (i.e. consisting of more than 10⁸ cells). To study the composition of these grafts in a small animal model, FACS technology was used to sculpt mouse-sized grafts consisting of less than 10⁶ cells.

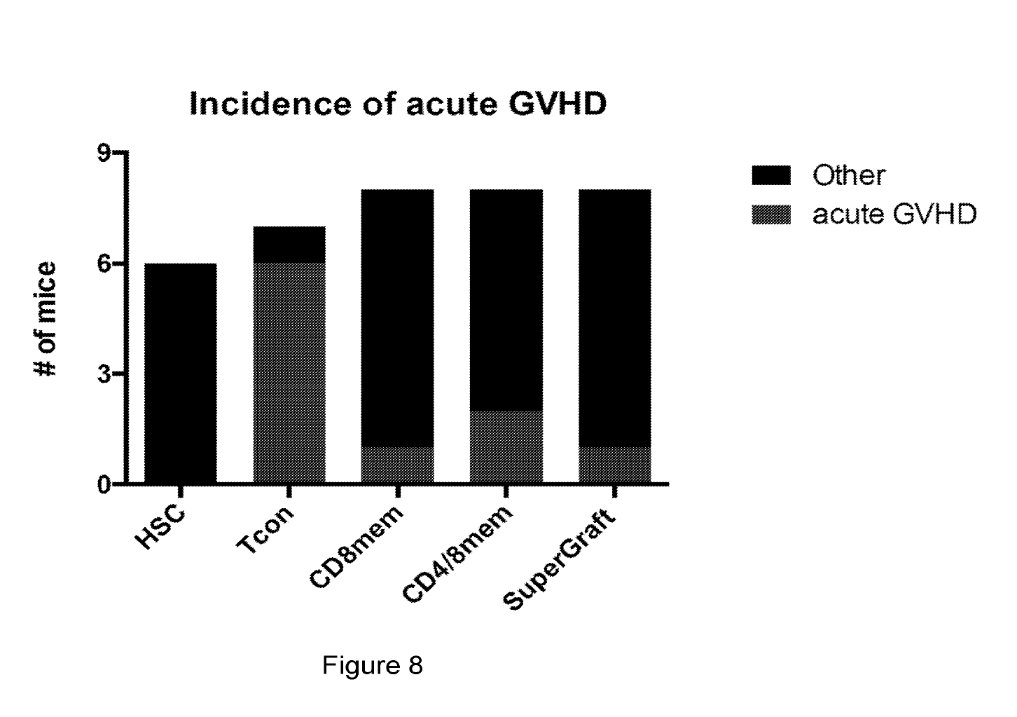

To examine the GVHD induction in sculpted grafts, specific lymphocyte subtypes were transplanted across a major mismatch barrier: from C57Bl/6 mice into myeloablated Balb/c mice (FIG. 8). To monitor anti-tumor potential of different immune compositions, mice were also challenged with a syngeneic tumor cell line (J774) carrying a stable GFP luciferase transgene. Specifically, 8-wk old female Balb/c recipients were irradiated with 800 rad and injected intravenously 24 hours later with 5000 J774 GFP-Luciferase cells. 24 hours later, mice received intravenous injections of the following lymphocyte populations.

To model bone marrow transplantation in humans, HSCs (ckit+Sca-1-Lin-) from bone marrow were transplanted alongside the indicated T cells fractions purified from the donor spleens (Table 4). CD4⁺ and CD8⁺ cells were first MACS enriched from the spleen either used directly to model convention T cells (Tcon) or further purified by FACS using CD19, CD11c, and B220 as negative selection lineage marker and CD4⁺CD44⁺ or CD8⁺CD44⁺ to denote memory cells. CD4⁺CD25^{hi} was used to identify Tregs and CD4⁺CD1d(PBS57 loaded)-tetramer⁺ was used to identify iNKT cells.

TABLE 4
Cell composition of engraftment populations.

| Graft- | | CD4/8 | | | Treg/ |
|---|---|---|---|---|---|
| Type | HSC | macs | CD8mem | CD4mem | iNKT |
| HSC | 10K | — | — | — | — |
| Tcon | 10K | 4 × 10⁶ | — | — | — |
| CD8mem | 10K | — | 5 × 10⁴ | — | — |
| CD4/8mem | 10K | — | 5 × 10⁴ | 2 × 10⁵ | — |
| Supergraft | 10K | — | 5 × 10⁴ | 2 × 10⁵ | 2 × 10⁵/ |
| | | | | | 1.5 × 10⁵ |

The animals were weighed regularly and GVHD was measured. Acute GVHD is defined by weight loss >10% of initial body weight within 3 weeks of transplant followed by recovery. The Tcon, which models the components of a bone marrow transplant currently administered in the clinic, elicits a vigorous GVHD response. (FIG. 8). The HSC transplant entirely prevents GVHD, whereas GVHD is markedly reduced in the purified lymphocyte grafts. (FIG. 8).

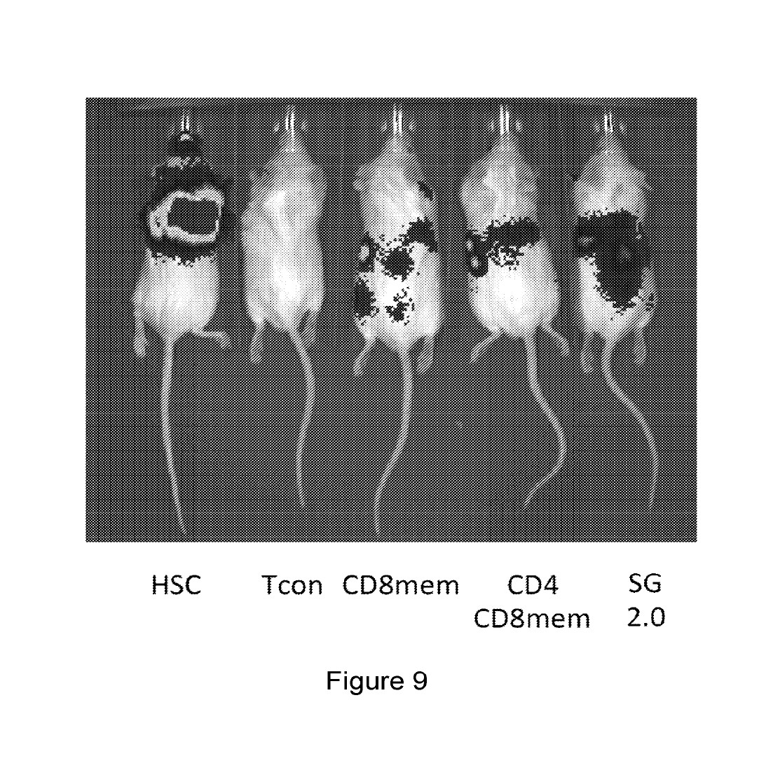

At 3 weeks post-transplant, the mice given HSCs were overrun with the tumor, whereas mice injected with the purified lymphocytes showed a reduced tumor burden (FIG. 9). The mice receiving the conventional T cells show an incredibly low tumor burden, but also developed high incidences of GVHD. Therefore, selection of the right types of immune cells can direct an efficient GVL response without eliciting GVHD.

Although preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

What is claimed is:

1. A method for sorting a heterogeneous starting population of cells having a plurality of phenotypes, the method comprising:
(a) loading a micropore array with a starting population of cells;
(b) imaging the micropore array to identify individual pores comprising cells having a phenotype of interest;
(c) extracting cells having a phenotype of interest from the individual pores by directing electromagnetic radiation at a radiation absorbing material associated with the pores, and;
(d) collecting extracted cells having a phenotype of interest, wherein the scanning, imaging, and extracting steps sort between 2×10⁹ and 4×10⁹ micropores within 120 minutes.

2. The method of claim 1, wherein one or more phenotypic markers in cells of the starting population is detected with binding agents capable of detecting a phenotype or phenotypes of interest.

3. The method of claim 2, wherein a binding agent is an antibody or antibodies.

4. The method of claim 1, wherein the micropore array is enclosed in a sorting cartridge comprising a sterile housing.

5. The method of claim 4, wherein the housing prevents the starting population of cells or the extracted cells from contacting of a sorting instrument during use.

6. The method of claim 1, wherein the starting population of cells is comprised of bone marrow cells, peripheral hematopoietic cells, differentiated ES cells, iPSCs, or genetically modified cells.

7. The method of claim 1, wherein the extracted cells comprise a population suitable for allogeneic or autologous transplantation into a subject.

8. The method of claim 1, wherein the starting population of cells comprises heterogeneous peripheral hematopoietic cells, and the extracted cells comprise a population of purified hematopoietic cells with reduced naïve T-cells.

9. The method of claim 8, wherein the extracted cells comprise a population of purified hematopoietic cells consisting of less than 0.0014% naïve T-cells.

10. The method of claim 8, wherein the population of purified hematopoietic cells result in reduced or undetectable incidence of graft-versus-host-disease when transplanted into a subject.

11. The method of claim 1, wherein the starting population of cells comprises a heterogeneous population containing tumorigenic or teratogenic cells, and the extracted cells comprise a population of purified cells free substantially or completely lacking tumorigenic or teratogenic cells.

12. The method of claim 1, wherein the starting population of cells comprises a heterogeneous population of differentiated Embryonic Stem Cells, or a heterogeneous population of differentiated induced Pluripotent Stem Cells, and the extracted cells comprise a homogenous population of differentiated cells.

13. The method of claim 1, wherein the starting population of cells comprises a heterogeneous population of cells subjected to genetic modification, and the extracted cells comprise a homogenous population of cells subjected to genetic modification.

14. The method of claim 1, wherein at least 10⁵ cells are scanned and imaged simultaneously.

15. The method of claim 1, wherein the scanning, imaging, and extracting steps are performed at a rate of at least about 500×10⁵ cells/sec.

16. The method of claim 1, wherein less than or equal to 1 cell is loaded into to each pore of the micropore array.

17. The method of claim 1, wherein the micropore array further comprises particles comprising a radiation absorbing material adhered to the interior walls of the pores.

18. The method of claim 1, wherein extracting cells by directing electromagnetic radiation at radiation absorbent material associated with the pores comprises a 1 nsec, 90 μJ laser pulse.

19. The method of claim 1, wherein the rapid extraction is achieved by using a polygon scanning system.

20. A system comprising:
(a) micropore array;
(b) an electromagnetic radiation source,
(c) a rotating polygon mirror,
(d) an F-theta lens, wherein the mirror and the lens scan the focus of the electromagnetic radiation source at micropores of the array at a rate of greater than 150,000 micropores per second, and
(e) a detector for detecting electromagnetic radiation from the pores of the array
